(19) United States
(12) Patent Application Publication
(10) Pub. No.: US 2010/0034859 A1
(43) Pub. Date: Feb. 11, 2010

(54) Method for making an implantable biocompatible material with mixed Pseudo-crystalline lattice and material obtainable by said method

(76) Inventor: Christian Mai, Lyon (FR)

Correspondence Address: COOPER & DUNHAM, LLP, 30 Rockefeller Plaza, 20th Floor, NEW YORK, NY, 10112, US

(21) Appl. No.: 12/308,215

(22) PCT Filed: Jun. 11, 2007
(86) PCT No.: PCT/FR2007/000966

§ 371 Date: Sep. 28, 2009

(30) Foreign Application Priority Data
Jun. 9, 2006 (FR) 06 05151
Aug. 3, 2006 (FR) 06 07139
Feb. 5, 2007 (FR) 07 00796

(51) Int. Cl. A61K 9/00; A61K 47/02; A61P 43/00

(52) U.S. Cl. 424/422; 514/769

(57) ABSTRACT

The invention concerns a method for making a biocompatible material implantable in a human or animal body including: a step (a) of dispersing at least one biocompatible substance in a solvent, to obtain an intermediate solution; a step (b) of condensation of said intermediate solution to obtain an amorphous condensate of said biocompatible substance; a step (c) of mixing the biocompatible substance with at least one agent for nucleating said biocompatible substance; a step (d) of activating the nucleating agent to generate the development, within said amorphous condensate, a mixed pseudo-crystalline lattice consisting of both the biocompatible substance and the nucleating agent, so as to obtain a biocompatible material at least partly crystallized. The invention also concerns biocompatible materials implantable in a human or animal body.

### TECHNICAL DOMAIN

The present invention belongs to the general technical domain of biocompatible materials intended for implantation (e.g. by subcutaneous injection or surgery) inside human or animal bodies for therapeutic and/or aesthetic purposes.

The present invention concerns in-particular a manufacturing process for biocompatible material implantable in humans or animals, as well as a biocompatible material that may be obtained using this process, and preferably obtained directly by the said process.

The invention primarily concerns a biocompatible material, as well as the related manufacturing process, intended for use in plastic surgery and/or reparative surgery, whether for tissue volume increase (e.g. increased cheek and chin volume, lip remodelling, correction of defects subsequent to rhinoplasty) or tissue filling (e.g. filling of wrinkles, small wrinkles and grooves appearing in the skin, particularly in the facial area). However, this invention is not limited to the production of material used in aesthetic surgery alone, and also concerns the production of material that may be used in functional and reparative surgery, for example in the following fields: bone filling, orthodontic surgery, neurosurgery, orthopaedic surgery, urological surgery and ophthalmology.

### PREVIOUS TECHNIQUE

A number of biocompatible materials for implantation in the human body or to fill empty areas of tissue or increase the volume of certain tissues are already in use.

In particular, there exist non-bioresorbable biomaterials, or materials having low and slow bioresorption properties (e.g. bioresorption over a period in excess of three years), such as coral and ceramics (e.g. hydroxyapatite) used in particular in bone surgery and dental surgery.

Such materials may be used to achieve substantial bone filling. However, these materials are not completely suitable for certain therapeutic and/or aesthetic procedures, and in particular treatments involving superficial subcutaneous injection of the material, for instance for wrinkle filling. Because of their relatively hard consistency, such materials may cause discomfort in patients, particularly where the material is implanted at the surface of sensitive areas such as the face. In addition, the long duration (or even absence) of biodegradability of these materials within the body may be a cause for concern, whether or not legitimate, regarding the long-term fate of such implants within the body, and such fears could dissuade patients from consenting to the use of these materials for aesthetic procedures such as wrinkle filling. Furthermore, a long period of bioresorption, and more particularly, absence of resorption, requires the conduct of long, complex and costly scientific and clinical studies before such materials may be marketed in order to verify their safety in use. Obviously, this increases the price of such materials.

Biocompatible materials are also available characterised by their rapid bioresorption by the human body, such as polymers like hyaluronic acid and a number of protein substances such as collagen.

Because of their rapid resorption within the body (which may be complete within a few months), these biomaterials do not produce very satisfactory aesthetic and/or functional results for a significant period of time. One consequence of their use, for instance in wrinkle filling, is that patients must undergo very frequent injections, with all the associated discomfort and risks. Clearly the duration of resorption of the above products may be increased, particularly that of hyaluronic acid, by means of additional treatments such as reticulation. However, treatment such as reticulation may be difficult to implement in repeated and reliable fashion (thereby increasing the price of these materials), and it may require the use of potentially toxic reticulant products.

In addition, there exists an acrylic cement intended for percutaneous injection in bone diseases (e.g. fractured bone or bone invaded by metastases) in order to consolidate and mechanically reinforce the affected bone. This cement is prepared on the operating table prior to injection. It consists of a mixture of a liquid monomer and a polymer powder, which produce an injectable paste that gradually hardens as a result of polymerisation reaction (hardening of the cement).

Nevertheless, although it provides beneficial mechanical reinforcement of bone, this cement has the following disadvantages:
it is difficult to control the polymerisation reaction and the cement can harden too rapidly prior to injection;
use of this cement is restricted to certain indications (e.g. surgery after metastatic deterioration of a vertebra but prior to total rupture of the vertebra) because of the difficulty of injecting the cement;
the polymerisation reaction is exothermic (involving temperatures that may be fairly high, such as 80° C. in some cases), which presents a risk to adjacent tissue and may result in degradation of therapeutic substances present in the cement.

### DESCRIPTION OF THE INVENTION

The aims of the invention are thus to remedy the various disadvantages listed above and to propose a new manufacturing process for biocompatible materials suitable for implantation in the body of humans or animals that is easy to use and inexpensive to produce, while allowing simple and precise control of the degree of bioresorption of the said material.

Another aim of the invention is to provide a new manufacturing process for a biocompatible material implantable in the body of humans or animals using elementary biocompatible products as such and, in addition, which are inexpensive.

Another aim of the invention is to provide a new manufacturing process for a biocompatible material implantable in the body of humans or animals so as to obtain an especially homogeneous biomaterial.

Another aim of the invention is to provide a new manufacturing process for a biocompatible material implantable in the body of humans or animals resulting in a material that is particularly safe and comfortable for patients.

Another aim of the invention is to provide a new manufacturing process for a biocompatible material implantable in the body of humans or animals allowing especially simple and reliable control of the duration of bioresorption of the resulting material.

Another aim of the invention is to provide a new manufacturing process for a biocompatible material implantable in the body of humans or animals presenting therapeutic properties.

Another aim of the invention is to provide a new manufacturing process for a biocompatible material implantable in the body of humans or animals which, while presenting a long duration of bioresorption, is comfortable for patients when implanted under the skin, particularly when used for wrinkle filling.

Another aim of the invention is to provide a new manufacturing process for a biocompatible material implantable in the body of humans or animals which results in a material that is particularly easy to store, handle and shape.

Another aim of the invention is to provide a new manufacturing process for a biocompatible material implantable in the body of humans or animals having a precisely calibrated bioresorption rate and presenting safe and comfortable characteristics for patients while being especially effective and inexpensive.

The aims of the invention are achieved by means of a manufacturing process for a biocompatible material implantable in the body of humans and animals comprising:
a step (a) involving dispersion of at least one biocompatible substance in a solvent resulting in an intermediate solution,
a step (b) involving condensation of the said intermediate solution resulting in an amorphous condensate of the said biocompatible substance,
a step (c) involving mixing of the biocompatible substance with at least one nucleating agent for this biocompatible substance,
a step (d) involving activation of the nucleating agent to generate development within the said amorphous condensate of a mixed pseudo-crystalline lattice formed from the biocompatible substance and the nucleating agent so as to obtain a biocompatible material that is at least partly crystallised.

The aims of the invention are also achieved by means of a biocompatible material that is at least partly crystallised and implantable in the body of humans or animals and which comprises an amorphous condensate of a biocompatible substance and a nucleating agent for this biocompatible substance mixed with the latter in the amorphous condensate, a mixed pseudo-crystalline lattice formed by both the biocompatible substance and the nucleating agent being developed within the condensate.

The aims of the invention are also achieved by means of biocompatible material implantable in the body of humans or animals characterised by that it comprises a partly crystalline powder obtainable by a process according to the invention, the said powder being dispersed in an injection vector to form an injectable compound with the latter for tissue volume expansion and/or filling.

### SUMMARY DESCRIPTION OF DRAWINGS

Further advantages of the invention will be explained in detail in the following description and in the illustrations provided, purely for the purposes of explanation and not limited thereto, in which:

FIG. 1 shows the spectrum obtained by X-ray diffraction of an amorphous condensate.

FIG. 2 illustrates the spectrum obtained by X-ray diffraction of a material produced using the process described in the invention, starting from the amorphous condensate shown in FIG. 1; the spectra for natural bone and coral are also shown for reference.

### OPTIMAL IMPLEMENTATION OF THE INVENTION

The invention concerns a manufacturing process for a biocompatible material for implantation in the body of humans or animals for therapeutic and/or aesthetic purposes, and for such a material per se. In a preferred embodiment, the process governed by the invention comprises a manufacturing process for a biocompatible implantable material for tissue filling and/or volume increase, in particular for soft tissues such as skin.

The process described in the invention thus provides an advantageous manufacturing process for an implantable biocompatible material intended for use in one of the following applications: filling of wrinkles and grooves in the skin, particularly in the face (wrinkles between the eyebrows, wrinkles around the mouth, wrinkles at the sides of the eyes, expression lines, nasal grooves), treatment of defects following rhinoplasty, through increased tissue volume, remodelling of the lips, particularly vermilion tissue, increased cranial-facial volume (in particular, increased chin and/or cheek size), remodelling of the philtrum, it being understood that this list is not limiting. In particular, the process described in the invention is a manufacturing process for an injectable biocompatible material for wrinkle filling, i.e. a biomaterial intended for injection using a syringe under the skin of the patient in order to correct wrinkles or grooves, particularly on the surface of the face. The process described in the invention is, however, not limited to the creation of an injectable biocompatible material for plastic surgery, but may also constitute, depending on other methods of production covered by the framework of the invention, a manufacturing process for an implantable biocompatible material intended for use in one of the following applications: bone filling (particularly vertebroplasty), orthodontic surgery, neurosurgery, orthopaedic surgery, urological surgery (in particular, treatment of vesicoureteral reflux and treatment of female urinary incontinence) treatment of the genital apparatus (in particular, treatment G-spot disorders by injection into the genital wall), ophthalmic surgery, vocal cord plasty, radiolabelling of biological tissue; once again this list may not be considered as restrictive. In general, the manufacturing process described in the invention results in the production of a class of implantable biocompatible materials whose properties, particularly of bioresorption, may be readily adapted to meet the constraints associated with a number of therapeutic and/or aesthetic indications, certain of which are cited above.

The manufacturing process described in the invention involves a step (a) comprising dispersion of at least one biocompatible substance in a solvent, and the said step (a) thus results in an intermediate solution.

In other words, in step (a), a biocompatible substance in the form of a solute is mixed with a solvent of this solute in order to dissolve the biocompatible substance in the solvent. Preferably, the solvent used in step (a) is a liquid. The solvent used in step (a) should preferably contain an alcohol. The solvent used in step (a) should preferably contain ethyl alcohol and/or methyl alcohol since these two alcohols result in a final product having excellent biocompatibility. The solvent may consist entirely of ethyl alcohol or methyl alcohol. Nevertheless, the solvent should preferably not consist entirely of an alcohol but should also contain water mixed with this alcohol. Preferably, the solvent used in step (a) thus consists of an aqueous solution in an ethyl and/or methyl medium. To this aqueous solution in an ethyl or methyl medium is added the biocompatible substance, which at this stage is for instance in the form of dry powder or a solution.

Preferably, step (a) comprises a sub-step (a′) involving stirring of the solvent in order to ensure homogeneity of the dispersion and dissolution of the biocompatible substance in the solvent. The solvent may also be agitated before mixing with the biocompatible substance, and agitation may be continued during and after placing the biocompatible substance in the solvent. Alternatively, but still within the scope of the invention, the biocompatible substance may be placed in the resting solvent, and the solvent/biocompatible substance mixture may then be stirred to ensure homogeneity of the mix. Preferably, during stirring sub-step (a′), a vortex may be generated within the solvent so as to ensure thorough mixing of the biocompatible substance and the solvent with complete dissolution of the biocompatible substance in the solvent. Preferably, generation of the vortex within the solvent is achieved by means of a magnetic stirrer in the solvent spinning at high speed. Thus, using this preferential embodiment of the invention, a vortex is created in an aqueous solution in an ethyl or methyl medium comprising the solvent by means of high speed, magnetic stirring after which the biocompatible substance is added to the solvent with stirring in order to ensure homogeneous and uniform dissolution of the latter in the solvent. Obviously, homogeneity of dissolution of the biocompatible substance in the solvent may be achieved by means other than the generation of a vortex by magnetic stirring at high speed, with the main objective being to ensure that stirring of the solvent and biocompatible substance is adequate to guarantee proper dissolution of the biocompatible substance in the solvent.

To summarise, step (a) results in a highly homogeneous intermediate solution which in turn results in the production of a highly homogeneous final product.

The biocompatible substance intended for dispersal in the solvent during step (a) should be roughly crystalline.

For example, the biocompatible substance used in step (a) comprises at least calcium and/or at least a calcium derivative. The use of a biocompatible substance containing calcium is particularly useful, principally because of the excellent biocompatibility of calcium, which is a natural component of bone and teeth.

Preferably, the biocompatible substance used in step (a) mainly consists of calcium and/or at least a calcium derivative. For example, the biocompatible substance mainly comprises compounds containing calcium such as calcium nitrate and/or calcium carbonate and/or calcium chloride. It is especially desirable that the biocompatible substance should be composed chiefly of calcium carbonate and/or calcium nitrate. The biocompatible substance may obviously contain other compounds, either replacing calcium or added to calcium, such as biocompatible trace elements.

For example, the biocompatible substance may comprise, in addition to calcium-containing compounds, trace elements comprising magnesium and/or potassium and/or glucose and/or fluorine.

These trace elements have the advantage of offering good control and improving the biocompatible character of the finished product obtained through the use of the process governed by the invention. These trace elements may also be selected to activate certain physiological processes beneficial for biological tissue with which the biocompatible material obtained by the process covered by the invention is intended to come into contact. Preferably, these trace elements will thus be chosen as a function of the intended application, in accordance with their bioactivity on tissue, i.e. their ability to induce one or more specific physiological reactions within the said biological tissues.

Although it is particularly advantageous to use bioactive trace elements during step (a) or prior to this step, it is also feasible to introduce the said trace elements only at a later stage of the process, as specified below.

In general, the process covered by the invention thus comprises a step (i) of incorporation of at least one bioactive substance.

With a specific embodiment, step (a) involves the addition of calcium-containing compounds, (e.g. calcium carbonate or calcium nitrate), preferably supplemented with trace elements (e.g. containing magnesium, potassium, fluorine or glucose), in an aqueous solution in an ethyl or methyl medium subjected to agitation by high-speed magnetic stirring in order to ensure dissolution and complete mixing of the above-mentioned compounds to form the biocompatible substance in an aqueous solution of alcohol that acts as the solvent. This step (a) thus produces an intermediate solution comprising a solute (formed by the biocompatible substance) dissolved in the solvent.

The process covered by the invention also covers a step (b) involving condensation of the said intermediate solution, resulting in an amorphous condensate of the said biocompatible substance, with the said condensate thus containing the biocompatible substance in non-crystalline form.

Step (b) thus preferably follows step (a) and is distinct from the latter.

In summary, successive performance of steps (a) and (b) leads to the creation of an amorphous biocompatible substance from an initial biocompatible substance that may be crystalline, such as calcium carbonate or calcium nitrate for example.

Preferably, condensation step (b) involves precipitation of the said intermediate solution in order to obtain the amorphous condensate. Preferably, condensation step (b) comprises a sub-step (b′) involving addition to the said intermediate solution of an acid and/or base to trigger precipitation of the biocompatible substance. In other words, precipitation of the mixture of the biocompatible substance/solvent is triggered in step (b) by adding to the said mixture an acid (e.g. hydrofluoric acid) or a base, the type and quantity of which are selected as a function of the composition of the intermediate solution obtained in step (a). Preferably, precipitation step (b) is carried out without heat, i.e. at ambient temperature, for example at appreciably below 50° C.

Condensation step (b) thus results in the creation of an amorphous condensate, i.e. a non-crystalline substance, preferably in the form of a highly viscous paste. Obviously, use of a precipitation stage although preferable because of its simplicity and efficacy, is not absolutely necessary, and condensation may be achieved by other methods (e.g. evaporation).

At the end of step (b), residual solvent may exist alongside the amorphous condensate, i.e. step (b) may result in the creation of an amorphous condensate and a residual solvent. Consequently, the process governed by the invention preferentially involves a stage (h) after stage (b) in order to eliminate the residual solvent (for example by heat treatment) which may be present together with the condensate at the end of step (b). Preferentially, during step (h), the residual solvent/amorphous condensate mixture is heated to a temperature of between 50 and 300° C., and preferably between 100 and 200° C., in order to eradicate the residual solvent found with the condensate by means of evaporation.

The manufacturing process governed by the invention also comprises step (c) involving mixing of the biocompatible substance with at least a nucleating agent of this biocompatible substance. In other words, step (c) involves placing the biocompatible substance in contact with a nucleating agent specifically suited to the physicochemical properties of the said biocompatible substance in order to initiate a crystallisation reaction of the said biocompatible substance. Preferably, in particular where the biocompatible substance contains calcium carbonate or calcium nitrate, the nucleating agent will contain at least one metal oxide and/or non-metal oxide, and in this case will contain at least titanium oxide and/or zirconium oxide and/or silicium oxide.

The use of metal oxides and/or non-metal oxides such as those indicated above as nucleating agents for a biocompatible substance chiefly comprising calcium (or calcium derivatives) is particularly useful since it allows precise and simple control over the degree of crystallinity of the resulting final material, which also presents a high degree of biocompatibility, and thus great safety of use. Preferably, the nucleating agent is in a dispersed form, for example in a powder or liquid form, so as to enable a good, homogeneous and uniform mixture to be obtained with the biocompatible substance within the condensate.

Step (c) thus comprises initiation of crystallisation, with crystallisation itself being effectively performed as described below.

Step (c) involving addition of the nucleating agent may be carried out at different stages of the process governed by the invention.

For instance, step (c) involving the addition of the nucleating agent may usefully be performed before or during step (a), and prior to precipitation step (b), so that the intermediate solution obtained after step (a) contains the said nucleating agent. In this case, precipitation step (b) will be performed on an intermediate solution containing the solvent, the biocompatible substance dissolved in the solvent and the nucleating agent. Precipitation of the intermediate solution already containing the nucleating agent thus results in an amorphous condensate also containing the nucleating agent.

However, and still within the scope of the invention, step (c) may easily be performed before step (b), i.e. before an amorphous condensate is obtained, in order to ensure that the said amorphous condensate contains the nucleating agent. In this case, the nucleating agent should preferably be added directly to the amorphous condensate, preferably after step (h) comprising elimination of the residual solvent.

This produces an amorphous condensate of the biocompatible substance containing the nucleating agent, with the latter preferably being dispersed homogeneously throughout the condensate.

Thus, at the end of step (c), and regardless of when step (c) is completed during the process, the amorphous condensate will contain the nucleating agent, with the latter designed to trigger crystallisation of the condensate.

Step (c) is preferably conducted in such a way that the amorphous condensate contains between 1% and 80% nucleating agent by weight at the end of step (c). Preferably, step (c) should be conducted to ensure that the amorphous condensate contains between 10% and 60% by weight of nucleating agent at the end of step (c), with a preference for a range of 10-40% since this ensures a semi-crystalline material with a bioresorption duration of between 1 and 5 years, which is particularly suited to certain applications in aesthetic medicine (e.g. bone filling, increased cranial-facial volume or wrinkle filling).

The process governed by the invention also comprises step (d), which is subsequent to steps (b) and (c), and preferably to step (h) involving elimination of the residual solvent, and this step involves activation of the nucleating agent in order to trigger the development within the said amorphous condensate of a mixed pseudo-crystalline lattice formed both by the biocompatible substance and the nucleating agent, so as to obtain a biocompatible material that is at least partly crystallised.

In other words, during step (d) involving activation of the nucleating agent, the amorphous condensate containing the dispersed nucleating agent undergoes a process leading to crystalline growth within the initially amorphous condensate, beginning with germination nuclei consisting of the nucleating agent dispersed in the amorphous condensate. The concentrations of biocompatible substance and nucleating agent are selected in relation to one another in order to ensure that the crystallisation occurring within the amorphous condensate corresponds to the development of a skeleton (pseudo-crystalline lattice) made up of atoms of the nucleating agent and atoms of the biocompatible substance, in practically equal proportions (mixed lattice), with the said atoms linking to one another to form the said skeleton.

Activation step (d) thus allows the generation of a crystallisation phenomenon adjacent to the nucleating agent and propagation of this crystallisation phenomenon within the condensate, which was initially completely amorphous.

Step (d) may thus be considered as a step involving crystallisation of the amorphous condensate, ensuring that the said initially amorphous condensate is transformed into an at least partially crystalline biocompatible material. This crystallisation phenomenon results in particular, as indicated above, in incorporation of the nucleating agent in the crystalline lattice formed in the condensate of the biocompatible substance, ensuring that the biocompatible material obtained after step (d) is not simply a crystalline form of the initial biocompatible substance, but is rather a new material whose pseudo-crystalline lattice includes the nucleating agent. Thus, if the process governed by the invention is carried out using the biocompatible substance that includes calcium phosphate (or calcium carbonate), the biocompatible material obtained at the end of step (d) does not comprise calcium phosphate (or calcium carbonate) but rather a new substance containing calcium, at least partially crystalline and whose pseudo-crystalline lattice includes the nucleating agent. Similarly, the pseudo-crystalline lattice resulting from the process governed by the invention is not formed by the nucleating agent alone but rather by a combination of the nucleating agent and the biocompatible substance (mixed lattice). This mixed character of the resulting lattice, which is in fact partly responsible for the pseudo-crystalline nature of the lattice, allows ready and precise manipulation of the degree of pseudo-crystallinity of the condensate and thus its degree of bioresorbability.

Step (d) is preferably conducted in such a way as to ensure that the said biocompatible material obtained at the end of step (d) is only partly crystallised, i.e. it does not present an entirely crystalline (or pseudo-crystalline) structure. In other words, a fraction of the final biocompatible material is amorphous, while the remaining fraction is crystalline (or pseudo-crystalline), and this crystalline fraction corresponds to the degree of crystallinity of the material, which in any case is below 100%.

However, still within the scope of the invention, nucleating agent activation step (d) may be conducted in such a way as to ensure that the resulting biocompatible material is entirely crystallised (or pseudo-crystallised).

The invention is thus based in particular on the (pseudo) crystallisation of an amorphous material obtained as indicated above, the degree of crystallinity of which is directly associated (as demonstrated in the studies conducted by the applicant) with the duration of bioresorption of the resulting material once implanted in the body of a human or animal. The invention thus allows the production of a biomaterial having a controlled level of crystallinity.

The invention thus provides a very simple means of obtaining materials that are rapidly bioresorbable (duration of resorption less than one year), semi-permanent (duration of resorption between one and two years) or resorbable in the longer term (duration of resorption greater than two years), or in the very long term (duration of resorption greater than five years).

In order to meet the requirements of certain applications for which the invention is preferentially intended, namely wrinkle filling and increased tissue volume, which require a relatively long resorption time of between one and three years, step (d) should preferably be conducted in such a way as to ensure that the resulting biocompatible material at the end of step (d) has a crystallinity level of between 10% and 80% and preferably between 30% and 60%. Such levels of crystallinity are also particularly suitable for bone filling procedures.

The invention also concerns, independently, a process for the production of a biocompatible material implantable in the body of humans or animals that comprises a step involving partial crystallisation of an amorphous condensate, resulting in the production of a partially crystallised biocompatible material.

Preferably, step (d) is conducted so as to ensure that the said biocompatible material obtained at the end of step (d) has a level of crystallinity corresponding to a duration of bioresorption of between 12 and 18 months, and preferably between 15 and 18 months. This type of material is particularly well suited to wrinkle filling and/or increasing tissue volume.

Initiation of crystallisation in two steps (with the addition of a nucleating agent followed by activation of this nucleating agent), which allows precise control over this crystallisation and effective and rapid crystallisation, with excellent control over the final crystallinity level and thus over the duration of bioresorption of the material, also constitutes an independent invention per se. In other words, the invention also concerns, independently of the other characteristics described in the foregoing text, a manufacturing process for a biocompatible material implantable in the body of humans or animals comprising on the one hand a mixing step, possibly within a condensate of a biocompatible substance and a nucleating agent for this biocompatible substance, and on the other hand, an activation stage for the nucleating agent in order to trigger crystallisation of the biocompatible substance, resulting in a biocompatible material that is at least partly crystallised, and preferably only partly crystallised.

Preferably, activation step (d) comprises heating of the amorphous condensate containing the nucleating agent. In other words, in this particularly advantageous variant of the invention, crystallisation is obtained by adding one or more nucleating agents, preferably containing a metal oxide, to the amorphous condensate and with heat treatment of the condensate/nucleating agent mixture. For example, activation step (d) involves heating the amorphous condensate containing the nucleating agent to a temperature of between 35° C. and 1,000° C., but preferably to a temperature of between 300° C. and 900° C.

With this advantageous embodiment, the degree of crystallinity of the biocompatible material obtained at the end of step (c) is dependent on:
The concentration of nucleating agent
The temperature and duration of heat treatment for activation allowing the appearance and propagation of crystallisation within the condensate containing the nucleating agent.

Using this preferred embodiment governed by the invention, it is thus simple to precisely control the degree of crystallinity, and thus the degree of bioabsorption, of the final material obtained by selecting a suitable quantity of nucleating agent and a suitable activation temperature.

For example, an amorphous condensate containing calcium with between 10 and 20% in weight of nucleating agent containing metal oxide, heated to a temperature of between 300° C. and 700° C., results in a biomaterial with a degree of crystallinity of between 30 and 50%.

The same condensate, this time containing between 30 and 40% of the nucleating agent, when heated to a temperature of between 300° C. and 700° C. results in a biomaterial with a degree of crystallinity in excess of 50%.

The invention thus provides a particularly advantageous method of conferring a controlled level of crystallinity upon a substance, despite the fact that the latter is initially completely crystalline. The initially crystalline substance is in fact rendered amorphous by subjecting it to steps (a) and (b), after which it is re-crystallised in a controlled fashion by means of steps (c) and (d). The invention thus concerns, both in itself and in independent fashion, a manufacturing process for a biocompatible material for implantation in the body of humans or animals comprising on the one hand a step in which an initially crystalline biocompatible substance is made amorphous in order to produce an intermediate amorphous substance (preferably by dissolution and precipitation, although other techniques may be used such as extremely low-temperature treatment or projection at supersonic speed for example), and on the other hand, a step involving crystallisation of the amorphous intermediate substance resulting in a partly crystallised biocompatible material, with the crystallisation step being carried out preferably by the addition of a nucleating agent and activation of this nucleating agent by heating.

As indicated above, the process governed by the invention preferably involves a step (i) with incorporation of at least one bioactive substance (i.e. a substance able to activate at least one beneficial physiological process), in order to ensure that the said biocompatible material obtained at the end of step (d) contains the said bioactive substance.

In particular, the invention independently concerns a manufacturing process for a biocompatible material implantable in the body of humans or animals and comprising:
a step (a) involving dispersion of at least one biocompatible substance in a solvent resulting in the production of an intermediate solution,
a step (b) involving condensation (preferably through precipitation) of the said intermediate solution resulting in an amorphous condensate of the said biocompatible substance,
a step (c), involving mixing of the biocompatible substance with at least a nucleating agent of this biocompatible substance,
a step (d), involving activation of the nucleating agent to trigger development within the said amorphous condensate of a mixed pseudo-crystalline lattice formed by both the biocompatible substance and the nucleating agent so as to obtain a biocompatible material that is at least partly crystalline,
a step (i), involving incorporation of at least one bioactive substance, so that the said biocompatible material obtained at the end of step (d) contains the said bioactive substance.

The bioactive substance preferably contains one or more of the following elements: selenium, copper, zinc, strontium.

For example, in the case of a biocompatible material obtained using the process governed by the invention intended for implantation in bone, it is preferable for the said material to incorporate strontium, which favours proliferation of osteoblasts thereby enhancing bone filling. Where the biocompatible material resulting from the process governed by the invention is intended for implantation under the skin, for example for wrinkle filling, it is advantageous that the said material should incorporate selenium, copper and zinc, all three of which induce cellular activation of fibroblasts, promoting natural collagen production and thus tissue filling.

Step (i), involving incorporation of a bioactive substance, should preferably be conducted at the latest during step (d), and preferably before the said step (d). Using this preferential embodiment, crystallisation occurs within the amorphous condensate containing the bioactive substance, and thus at the end of step (d) a material is produced that includes a crystalline lattice within which the bioactive substance is contained. With this particularly advantageous embodiment, the invention thus allows the production of a biomaterial comprising a matrix within which the bioactive elements are trapped and dispersed homogeneously. Following implantation, this material is thus able to release the bioactive substance in a controlled, gradual and prolonged fashion throughout the duration of bioresorption, thereby optimising its efficacy. In conclusion, this material allows the creation of contact between the patient cells to be treated and a bioactive agent, the duration and rapidity of action of which are determined by the characteristics of the matrix. Thus the speed of resorption of the material and the bioavailability of the trace elements forming the bioactive substance are determined by the degree of crystallinity (ratio between the quantity of crystalline phase to the total quantity) of the material, while the degree of porosity (number and size of interstices present in the crystalline lattice) of the latter governs its integration by surrounding tissues.

However, still within the scope of the invention, it is entirely possible that step (i) may be performed after step (d), for example by simply mixing the biocompatible substance with the material obtained at the end of step (d).

The process governed by the invention advantageously comprises, totally distinctly and separately from the possible inclusion of step (i), step (j), involving incorporation of at least one therapeutic substance, in order to ensure that the resulting biocompatible material obtained at the end of step (d) contains the said therapeutic substance.

“Therapeutic substance” is taken in this context to mean a substance or active medical compound that:
presents curative or preventative properties with regard to one or more human or animal disorders, or
is able to restore, correct or modify one or more organic functions in humans or animals, or
allows medical or veterinary diagnosis to be performed.

Preferably, the therapeutic substance used will contain one or more of the following products: chemotherapy agents, analgesics or antibiotics.

For example, for a biocompatible material obtained using the process governed by the invention and intended for implantation in a bone invaded by metastases, it is advantageous that the said material should include a chemotherapy agent and an analgesic. This material will then achieve not only a mechanical effect of consolidation and reinforcement of the bone, but also a therapeutic effect (treatment of the metastases and associated pain).

Using a special embodiment, step (j) involving incorporation of a therapeutic substance is carried out no later than step (d), and preferably before the said step (d). With this embodiment, crystallisation occurs within the amorphous condensate containing the therapeutic substance in such a way as to ensure that at the end of step (d) a material is obtained that includes a pseudo-crystalline lattice in which the therapeutic substance is included. In this case, the invention allows the production of a biomaterial comprising a matrix containing therapeutic substances that are trapped and dispersed homogeneously throughout.

Using an alternative and preferred embodiment, step (j) is carried out after step (d), i.e. after the crystallisation process. In this case, step (j) may for example be carried out by means of impregnation of the material obtained at step (d) using a pressurised solution. This impregnation process ensures penetration of the therapeutic substance into the pores of the material.

The invention thus allows dispersal of therapeutic substances within the matrix constituting the material obtained at the end of step (d), and these substances are bound to the matrix by weak (non-covalent) bonds thereby facilitating their release. Thus, following implantation, the material produced according to the invention is able to release the therapeutic substance in controlled, gradual and prolonged fashion throughout the duration of its bioresorption, thereby ensuring optimal efficacy of the therapeutic substance. In summary, this material ensures contact between the patient's cells requiring treatment and an active pharmacological substance whose duration and rapidity of action are determined by the characteristics of the matrix. Thus, the rate of resorption of the material and the bioavailability of the trace elements forming the bioactive substance are determined by the level of crystallinity (the ratio of quantity of crystalline phase to total quantity) of the material, while the levels of porosity (number and size of interstices present in the crystalline lattice) of the latter govern its integration by adjacent tissue. The invention thus provides control over the speed and time of release of the medical substance, thereby avoiding massive release of the medicine, and because of the filling material used, no exothermic reaction is generated, resulting in ready control of solidification and viscosity, which are not dependent on any polymerisation reaction.

Using an additional facet of the invention, it is possible to mix the therapeutic substance with the condensate obtained in step (b) without proceeding to steps (c) and (d).

The process covered by the invention also has the advantage of step (e) involving pulverisation of the biocompatible material obtained at activation step (d), and the said step (e) results in production of a biocompatible powder. The granule size of this powder, which may be macroscopic, microscopic or nanoscopic, is based on the final intended use of the said powder. The process governed by the invention also includes a step (f) involving formulation of the biocompatible powder, preferably by fritting and/or compacting. For example, after pulverisation of partly crystallised biomaterial obtained at step (d), this powder is compacted and fritted at a temperature in excess of 900° C., resulting in a block of material that may be subsequently cut or shaped for reparative (orthopaedic) surgical applications in traumatology or orthodontic surgery.

In addition, instead of being fritted the biocompatible powder may be mixed with a polymer or an inorganic compound in order to obtain a bone cement that may be used for example in bone oncology (vertebroplasty).

The biocompatible powder may also be mixed with a suitable vector allowing it to be injected subcutaneously by syringe in order to carry out filling and/or increase soft tissue volume. In this case, the process involves step (g) comprising suspension of the said biocompatible powder in an injection vector, and the said step (g) results in an injectable compound. For example, the injection vector may comprise a solution of hyaluronic acid. For instance, powder containing calcium obtained in step (e) may be mixed with a solution (e.g. hyaluronic acid solution) or a gel (e.g. a gel comprising sodium carboxymethylcellulose, glycerine and water), thereby allowing easy introduction of the product into the patient's body by injection.

The invention also concerns, per se and independently, a manufacturing process for a biocompatible material injectable in the body of humans or animals, for example to form a product for increasing tissue volume and/or filling (such as wrinkle filling products), and this process comprises:
a manufacturing step or a step involving supply of an injection vector, which for example comprises a viscous solution of a resorbable biocompatible material,
a manufacturing step or a step involving supply of a powder of a partially crystallised biocompatible material (presenting a level of crystallinity between 5 and 80% for instance, and preferably between 10 and 60%), with the said powder obtained for example by means of the process described above,
a mixing step for the said powder with the injection vector in order to place the powder in suspension in the vector.

Preferably, the level of crystallinity of the powder is such that the said powder presents a duration of bioresorption of between 12 and 18 months, and preferably of 15 months. The powder is preferably formed from particles having a diameter of between 5 and 300 micrometers, and preferably between 10 and 200 micrometers.

The invention also concerns per se a biocompatible material at least partially crystallised and implantable in the body of humans or animals, and comprising an amorphous condensate of a biocompatible substance and a nucleating agent for this biocompatible substance mixed with the latter in an amorphous condensate, a mixed pseudo-crystalline lattice formed by both the biocompatible substance and the nucleating agent being developed within the condensate. Preferably, and as indicated above, the biocompatible substance consists chiefly of calcium and/or at least one calcium derivative, while the said nucleating agent preferably contains at least one metal oxide.

This biocompatible material implantable in the body of humans or animals, which is preferably only partly crystalline, may be obtained by the process according to the invention described above, and is preferably obtained by this process.

The invention concerns in particular an implantable biocompatible material, preferably partly crystalline and preferably obtained by the process governed by the invention, and having specially adapted physicochemical properties designed to ensure that the said material may be used in one or other of the following applications: bone filling, dental surgery, neurosurgery, orthopaedic surgery, urological surgery (vesicoureteral reflux and female urinary incontinence), radio labelling of tissues, vocal chord repair, cranial-facial volume increase, aesthetic surgery and in particular, filling of wrinkles and grooves in the skin, and ophthalmology; this list is not restrictive.

The invention also concerns, per se, a biocompatible material implantable in the body of humans or animals and comprising a partly crystalline powder that may be obtained by the process according to the invention and preferably obtained by this process, with the said powder being dispersed in an injection vector to form an injectable composite with the latter for tissue filling and/or volume increase.

For example, in the case of an injectable compound intended for wrinkle filling and/or tissue volume increase, the level of crystallinity of the powder obtained by the process governed by the invention shall be selected so as to confer on the said powder a duration of bioresorption (biodegradability within the body) of between 12 and 18 months, and preferably between 15 and 18 months.

In particular, the invention concerns per se a biocompatible material for tissue volume increase and/or filling that is partly crystalline, regardless of its consistency (solid, liquid, paste, powder). The level of crystallinity of the said material should be between 5% and 80%, and preferably between 10% and 60%. This level of crystallinity is selected to ensure that the said material has a duration of bioresorption of between 12 and 18 months, and preferably 15 months. This duration of bioresorption provides a plastic and/or functional effect for a significant duration while ensuring complete biodegradation of the material within a reasonable time, and in any case under two years, which may be interpreted by patients as a guarantee of safety, since patients, rightly or wrongly, may be afraid of having a permanent implant placed in their body.

The invention concerns in particular, and per se, a biocompatible material injectable in the body of humans or animals, for example to form a product for volume increase and/or tissue filling (e.g. wrinkle filling products), and the said material comprises:
an injection vector, which for example comprises a viscous solution of a resorbable biocompatible substance,
a powder of partly crystallised biocompatible material (with a level of crystallinity for example of between 5% and 80%, and preferably between 10% and 60%), with the said powder being obtained for instance using the process described above,
   with the said powder being mixed with the injection vector in order to ensure that the powder is suspended in the vector. Preferably, the level of crystallinity of the powder is determined in such a way as to ensure that the said powder has a duration of bioresorption of between 12 and 18 months, and preferably of 15 months. The powder should be made up of particles with a diameter of between 5 and 300 micrometers, and preferably between 10 and 200 micrometers.

The following examples, provided solely for the purposes of illustration and in no way limiting, provide a more precise illustration of the invention.

### Example 1

Step (a):

A solvent is prepared by mixing water and ethyl alcohol (4 moles of water for 1 mole of alcohol). A biocompatible substance is also prepared having the following composition: calcium nitrate, magnesium chloride, potassium carbonate and sodium chloride.

Step (c) is carried out during step (a), i.e. to the above-mentioned biocompatible substance is added a nucleating agent consisting of silicium tetraethyl (tetraethyloctosilicate).

At the end of step (c), a powder having the following composition is obtained:
calcium nitrate: 55% by weight
magnesium chloride: 10% by weight
potassium carbonate: 2.5% by weight
sodium chloride: 2.5% by weight
tetraethyloctosilicate: 30% by weight.

The solvent (aqueous solution of ethyl alcohol) is placed in a container and a vortex is created and maintained in the solvent using a magnetic stirrer.

The powder obtained in step (c) above is then dispersed in the solvent with stirring, resulting in dissolution of the powder in the solvent.

An intermediate solution is thus obtained.

Step (b):

Phosphoric acid is added to the intermediate solution (sub-step (b′)) in sufficient quantity to obtain precipitation of the intermediate solution, resulting in an amorphous condensate co-existing with the residual solvent.

Step (h):

The residual solvent is eliminated by heat treatment at 150° C. (evaporation of the residual solvent).

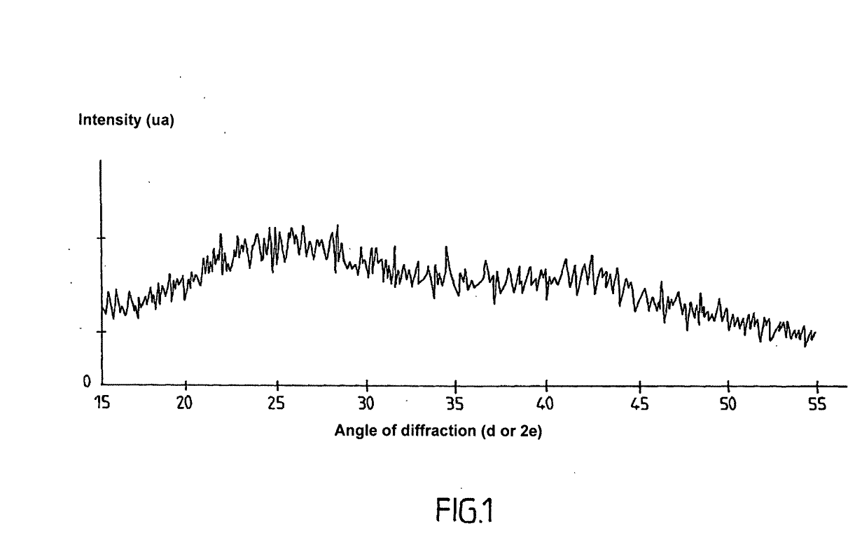

Thus, at the end of step (h), the amorphous condensate alone remains. The curve in FIG. 1 shows the structure of the amorphous condensate as determined by X-ray diffraction study.

Step (d):

The condensate is heated to a temperature of between 350° C. and 450° C. for around one hour, in order to induce partial crystallisation of the condensate.

This results in an implantable biocompatible material presenting a level of crystallinity of around 50%.

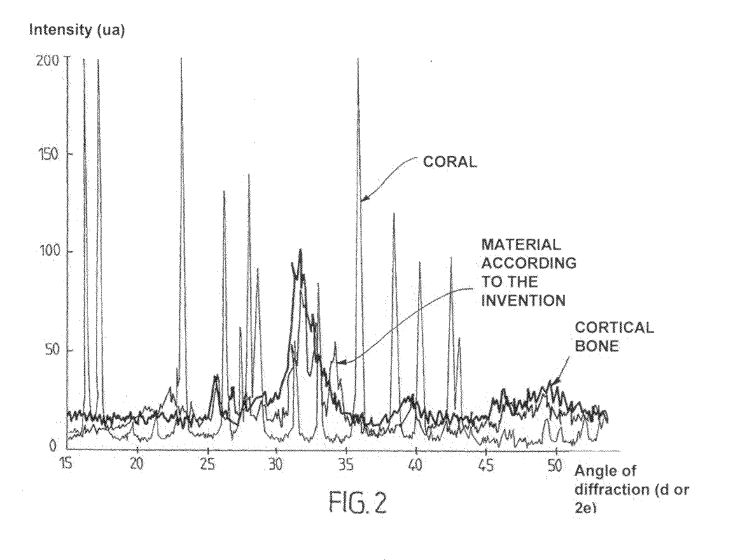

The X-ray diffraction study of the structure of this implantable biocompatible material yielded the curve in the graph shown in FIG. 2, and this graph also includes the spectral curve for cortical bone and the spectral curve for coral for reference. The graph in FIG. 2 shows that the spectrum for the material governed by the invention is extremely close to that of natural bone, in contrast with that of coral. Comparison of FIGS. 1 and 2 shows that structural proximity between the biocompatible material governed by the invention and natural bone is obtained as of steps (c) and (d).

The extreme proximity of the molecular structure of the material governed by the invention to that of natural bone ensures rapid, solid and durable reinforcement of bone lesions.

### Example 2

Example 2 follows exactly the same pattern as example 1, with the following two differences:

1) In step (a), the powder after step (c) has the following composition:
calcium nitrate: 45% by weight
magnesium chloride: 10% by weight
potassium carbonate: 2.5% by weight
sodium chloride: 2.5% by weight
tetraethyloctosilicate: 45% by weight.

2) In step (d), the condensate is heated to 750° C. for approximately two hours so as to induce partial crystallisation of the condensate. This produces an implantable biocompatible material with a crystallinity level of around 75%.

### Example 3

Example 3 is exactly the same as example 1, with the following two differences:

1) In step (a), the powder obtained at the end of step (c) has the following composition:
calcium nitrate: 45% by weight
magnesium chloride: 5% by weight
potassium carbonate: 2.5% by weight
sodium chloride: 2.5% by weight
tetraethyloctosilicate: 45% by weight.

2) In step (d), the condensate is heated to 850° C. for around two hours so as to induce partial crystallisation of the condensate. This results in an implantable biocompatible material with a level of crystallinity of approximately 90%.

### Example 4

The implantable biocompatible material obtained in examples 1 to 3 above is reduced to a fine powder, for example with a granule size of between 5 and 200 micrometers. This powder is suspended in a viscous solution of hyaluronic acid to form the injection vector. This results in an injectable compound intended for use in plastic and aesthetic surgery (tissue filling and/or volume increase).

### Example 5

The implantable biocompatible material obtained in examples 1 to 3 above is reduced to a fine powder, for example with a granule size of between 5 and 200 micrometers. These fine particles are then immersed in a hyaluronic acid solution of non-animal origin for 24 hours. The particles are thus impregnated individually with a hyaluronic acid coating. The particles coated with hyaluronic acid are then dried and freeze-dried and are compacted under a pressure approximately 4000 bar. This results in a material intended for use in orthopaedic and orthodontic surgery.

### POTENTIAL INDUSTRIAL APPLICATIONS

An industrial application of the invention concerns the manufacture and use of a biocompatible biomaterial implantable in the body of humans and animals for therapeutic, aesthetic and/or surgical applications, particularly for tissue volume increase and filling.

1. A manufacturing process for a biocompatible material implantable in the body of humans or animals and comprising:
step (a), involving dispersion of at least one biocompatible substance in a solvent, resulting in an intermediate solution,
step (b), involving condensation of the said intermediate solution, resulting in an amorphous condensate of the said biocompatible substance,
step (c), involving mixing of the biocompatible substance with at least one nucleating agent of this biocompatible substance,
step (d), involving activation of the nucleating agent to trigger development within the said amorphous condensate of a mixed pseudo-crystalline lattice formed by both the biocompatible substance and the nucleating agent so as to obtain a biocompatible material that is at least partly crystallised.

2. Process as described in claim 1 characterised by the fact that step (b) involving condensation includes an operation to precipitate the said intermediate solution in order to obtain the said amorphous condensate.

3. Process as described in claim 1 characterised by the fact that it comprises a step (j) involving incorporation of at least one therapeutic substance, so that the biocompatible material obtained in step (d) includes the said therapeutic substance.

4. Process as described in claim 3, characterised by the fact that step (j), involving incorporation of the therapeutic substance is performed before step (d).

5. Process as described in claim 3, characterised by the fact that the therapeutic substance comprises one or more of the following substances: chemotherapeutic agent, analgesic, antibiotic.

6. Process as described in claim 1, characterised by the fact that the solvent used in step (a) contains an alcohol.

7. Process described in claim 6, characterised by the fact that the solvent used in step (a) comprises ethyl alcohol and/or methyl alcohol.

8. Process as described in claim 1, characterised by use of an aqueous solvent.

9. Process as described in claims 7, characterised by the fact that the solvent comprises an aqueous solution in an ethyl and/or methyl medium.

10. Process as described in claim 1, characterised by the fact that step (a) comprises a sub-step (a′) involving stirring of the solvent to ensure homogeneity of dispersion of the biocompatible substance.

11. Process as described in claim 10, characterised by the generation of a vortex within the solvent during stirring sub-step (a′).

12. Process as described in claim 1, characterised by the fact that the said biocompatible substance used in step (a) contains at least calcium and/or at least one calcium derivative.

13. Process as described in claim 12, characterised by the fact that the said biocompatible substance mainly consists of calcium and/or at least one calcium derivative.

14. Process described in claim 13, characterised by the fact that the said biocompatible substance mainly comprises calcium carbonate and/or calcium nitrate.

15. Process as described in claim 1, characterised by the fact that step (c) is carried out before or during step (a) in order to ensure that the intermediate solution contains the said nucleating agent.

16. Process as described in claim 1, characterised by the fact that step (c) is carried out after step (b) in order to ensure that the amorphous condensate contains the said nucleating agent.

17. Process as described in claim 1, characterised by the fact that step (c) is conducted so as to ensure that the amorphous condensate contains between 10 and 60% of nucleating agent by weight.

18. Process as described in claim 1, characterised by the fact that the said nucleating agent contains at least one metal oxide.

19. Process as described in claim 1, characterised by the fact that the said nucleating agent contains at least one non-metal oxide.

20. Process as described in claims 18, characterised by the fact that the nucleating agent comprises at least titanium oxide and/or zirconium oxide and/or silicium oxide.

21. Process as described in claim 1, characterised by the fact that step (d) involving activation comprises heating of the amorphous condensate containing the nucleating agent.

22. Process as described in claim 21, characterised by the fact that step (d) involving activation comprises heating of the amorphous condensate containing the nucleating agent to a temperature of between 35° C. and 1000° C.

23. Process as described in claim 22, characterised by the fact that step (d) involving activation comprises heating of the amorphous condensate containing the nucleating agent to a temperature of between 300° C. and 900° C.

24. Process as described in claim 1, characterised by the fact that the biocompatible substance intended for dispersal in the solvent during step (a) is roughly crystalline.

25. Process as described in claim 1, characterised by the fact that step (d) is conducted in such a way as to ensure that the biocompatible material obtained in step (d) is only partly crystallised.

26. Process as described in claim 25, characterised by the fact that step (d) is conducted in such a way that the said biocompatible material obtained in step (d) has a crystallinity level of between 10 and 80%, and preferably between 30 and 60%.

27. Process as described in claim 25, characterised by the fact that step (d) is conducted in such a way that the biocompatible material obtained at the end of step (d) has a crystallinity level corresponding to a bioresorption duration of between 12 and 18 months, and preferably between 15 and 18 months.

28. Process as described in claim 1, characterised by the fact that step (d) follows step (b).

29. Process as described in claim 1, characterised by the fact that the biocompatible material obtained at the end of step (d) does not contain calcium phosphate.

30. Process as described in claim 1, characterised by the fact that it comprises a step (e) involving pulverisation of the biocompatible material obtained in step (d) to produce a biocompatible powder.

31. Process as described in claim 30, characterised by the fact that it comprises a step (f) involving formulation of the said biocompatible powder, preferably by fritting and/or compacting.

32. Process as described in claim 36, characterised by the fact that it comprises a step (g) involving suspension of the said biocompatible powder in an injection vector, the said step (g) thus resulting in an injectable compound.

33. Process as described in claim 32, characterised by the fact that the injection vector comprises a solution of hyaluronic acid.

34. Process as described in claim 1, characterised by the fact that step (b) involving condensation comprises a sub-step (b′) involving addition to the said intermediate solution of an acid and/or a base in order to trigger precipitation of the biocompatible substance.

35. Process as described in claim 1, characterised by the fact that it comprises a step (h), subsequent to step (b), involving elimination, for example by heat treatment, of any residual solvent co-existing with the condensate after step (b).

36. Process as described in claim 1 characterised by the fact that it comprises a step (i) involving incorporation of at least one bioactive substance, so that the biocompatible material obtained at the end of step (d) includes the said bioactive substance.

37. Process as described in claim 36 characterised by the fact that step (j) involving incorporation of a bioactive substance is carried out at the latest during step (d).

38. Process as described in claim 36 characterised by the fact that the bioactive substance contains one or more of the following elements: selenium, copper, zinc, strontium.

39. Process as described in claim 1 characterised by the fact that it comprises a manufacturing process for an implantable biocompatible material intended for use in one of the following applications:
filling of wrinkles and grooves in the skin
treatment of defects following rhinoplasty
lip remodelling
increased cranial-facial volume
remodelling of philtrum
bone filling
orthodontic surgery
neurosurgery
orthopaedic surgery
urological surgery
ophthalmic surgery
vocal chord plasty
radiolabelling of biological tissues
treatment of G spot disorders.

40. Biocompatible material at least partly crystallised and implantable in the body of humans or animals, and comprising an amorphous condensate of a biocompatible substance and a nucleating agent for this biocompatible substance mixed with the latter in an amorphous condensate, a mixed pseudo-crystalline lattice formed by both the biocompatible substance and the nucleating agent being developed within the condensate.

41. Material as described in claim 40, characterised by the fact that the said biocompatible substance consists chiefly of calcium and/or at least one calcium derivative.

42. Material as described in claim 40, characterised by the fact that the said nucleating agent contains at least one metal oxide.

43. Material as described in claims 40, characterised by the fact that it is obtained by the process according to one of claims 1 to 38.

44. Biocompatible material implantable in the body of humans or animals characterised by the fact that it comprises a partly crystalline powder obtainable by a process according to claim 1, the said powder being dispersed in an injection vector to form an injectable compound with the latter for tissue filling and/or volume increase.

45. (canceled)
